(19) United States
(12) Patent Application Publication
(10) Pub. No.: US 2012/0087902 A1
(43) Pub. Date: Apr. 12, 2012

(54) Therapeutic Use Of Probiotics

(75) Inventors: Maria Rescigno, Milan (IT); Carmelina Loguercio, Napoli (IT); Enzo Grossi, Milano (IT)

(73) Assignee: BRACCO S.P.A., Milan (IT)

(21) Appl. No.: 13/378,031

(22) PCT Filed: Jul. 20, 2010
(86) PCT No.: PCT/EP2010/060457

§ 371 Date: Dec. 13, 2011

Related U.S. Application Data
(60) Provisional application No. 61/226,770, filed on Jul. 20, 2009.

(51) Int. Cl. A61K 35/74; C12Q 1/02; G01N 33/566; A61P 1/16

(52) U.S. Cl. 424/93.45; 435/29; 435/7.1; 435/7.92

(57) ABSTRACT

The involvement of intestinal microbiota in the initiation and perpetuation of inflammatory bowel disease is widely accepted. To reestablish the homeostasis of the gut the use of probiotics has been proposed, with however, limited clinical benefit. This invention relates to an assay based on the modulation of the immunological activity in DCs (dendritic cells) by probiotic strains, in particular L. paracasei and their use in protecting against inflammatory bowel and liver diseases.

### BACKGROUND ART

The intestine is home to trillions of commensal bacteria that participate to the digestive function but also help protecting the host from the aggression of pathogens (Ley R E et al. (2006) Cell 124: 837-848). Commensals are not ignored by the immune system but are tolerated presumably via a concerted action of epithelial cells and immune cells (Rescigno M et al (2008) Curr Drug Targets 9: 395-403). However, commensals can also be colitogenic as several colitis susceptible mice when reared under germ free conditions do not develop colitis (Elson C O et al. (2005) Immunol Rev 206: 260-276).

Given the dual role of bacteria—protective versus colitogenic—the use of probiotics as therapeutic agents has been proposed. Probiotics are considered as microorganisms that are beneficial to the host. Several classes of microorganisms, including bacteria and yeasts, are regarded as probiotics. Given the heterogeneity of probiotics, it is clear that not all the microorganisms may have the same effect on the host, but that each probiotic may have distinct activities. Hence for the correct use of probiotics as therapeutic agents it is important to know their precise activity. In this regards it has been shown that some strains of Lactobacillus (L. reuteri and L. plantarum 299v) or a combination of 8 different bacterial strains (VSL#3) can prevent, ameliorate or treat experimental colitis in IL10 deficient mice (Madsen K L et al. (1999) Gastroenterology 116: 1107-1114 and Schultz Met al. (2002) Inflamm Bowel Dis 8: 71-80). By contrast, VSL#3 or L. rhamnosus GG (LGG) were ineffective on a different type of experimental colitis (dinitrobenzene sulfonic acid, DNBS-colitis in Shibolet O et al. (2002) Inflamm Bowel Dis 8: 399-406).

The use of probiotics as therapeutic or preventive agents in IBD has long been proposed, with however limited clinical efficacy (Isaacs K and Herfarth H (2008). Inflamm Bowel Dis 14: 1597-1605). The major reason for this failure stands in the great variability of the immunomodulatory properties of probiotics, in physiologic conditions, once adsorbed at the intestinal level in the presence of other commensal bacteria.

Probiotics have been proposed in pouchitis and ulcerative colitis patients however their use in Crohn's disease (CD) is difficult to ascertain given the paucity of randomized double-blind clinical trials (Isaacs K and Herfarth H, see above). Presently, treatment of IBD may require, depending on the severity level of the disease, administration of anti-inflammatory drugs, such as prednisone, TNF-inhibitors or even more severe treatments, such as surgery.

The extreme variability of the immunomodulatory properties for a number of probiotics used up to now and the lack of a test for predicting their behavior in vivo, may explain why, i.e. the use of LGG as additive therapy, reduced instead of increasing the median time to relapse and increased, instead of reducing the incidence of relapse in children with C D Szajewska Het al. (2001) J Pediatr 138: 361-365. The same can be said for the use of LGG after surgery, that was shown to be not beneficial to CD patients (Kalliomaki M et al. (2001). Lancet 357: 1076-1079).

Therefore the use of a specific probiotic starin may result advantageous in IBD and other conditions of the gastrointestinal tract, only after the immunomodulatory properties are measured by an assay, such as the one proposed in the present invention, in the presence of other bacteria mimicking the presence of commensal bacteria in the gut and thus highly predictive of their behaviour in vivo.

### SUMMARY OF THE INVENTION

The present invention relates to a method for selecting a probiotic strain having bowel anti-inflammatory properties, which comprises the steps of:
co-incubating isolated Dendritic Cells (DCs) in the presence of an aliquot of a culture medium where a putative probiotic strain has been grown, together with a strong bacterial antigen,
detecting the production levels of at least a cytokine selected from the group consisting of: IL-12p70, TNF-α and IL-10 produce by DCs upon co-incubation,
selecting a probiotic strain where a decrease of IL-12p70 and TNF-α levels and substantially no alteration of IL-10 levels, with respect to the cytokine level measured in the absence of the putative probiotic, is detected.

Surprisingly, the immunomodulatory properties on human DCs cells and the ability to switch-off the immuno-response of a probiotoc strain selected according to this assay, have been found as well in the culture supernatant depleted of bacterial cells (conditioned culture medium).

The assay, carried out in vitro, is thus proposed to select probiotic strains or supernatants thereof, within Lactobacilli and Bifidus bacteria genera, the most commonly used commercially, actually beneficial in Inflammatory Bowel Diseases and even in the presence of commensal bacteria representing a further bacterial stimulus.

Interestingly, the results of this assay have been found to strongly correlate with the probiotic behaviour in vivo, in a murine model of induced colitis.

According to invention relates to the Lactobacillus paracasei strain CNCM I-1390 or supernatants thereof by oral administration for use as anti-inflammatory agents in the treatment or prevention of an inflammatory bowel disease (Crohn's disease, ulcerative colitis etc) and in the prevention or treatment of their recurrence.

Moreover the probiotic selected by this assay has been demonstrated as beneficial for treating and preventing chronic liver diseases in a mammal, in particular liver fibrosis due to chronic liver intoxication for drug assumption or metabolic disorders comprising administering to a mammal in need thereof an effective amount of a Lactobacilli paracasei strain (CNCM I-1390) or supernatant thereof in a oral composition.

### BRIEF DESCRIPTION OF THE DRAWINGS

FIG. 1. DCs incubated with different bacterial strains produce a distinct cytokine profile.

A. DCs were incubated or not with the reported live bacterial strains for 1 h in medium without antibiotics, washed and incubated for 23 h in medium with antibiotics. Culture supernatants were collected and tested for cytokine contents by ELISA. Each symbol represents a different DC donor. Red lines represent mean values. *, p<0.05; **, p<0.01.

S. typhim.: S. typhimurium; L. plant.: L. plantarum; L. parac.: L. paracasei.

B. To analyze the kinetic of cytokine production, DCs were incubated or not with the reported live bacterial strains for 1 h in medium without antibiotics, washed and incubated for 3-5 h in medium with antibiotics. Culture supernatants were collected and tested for cytokine release by ELISA. Error bars: standard deviations on values obtained on 4 different donors.

FIG. 2. Lactobacilli-Treated DCs have different ability to induce T cell proliferation and cytokine production.

A. T cell proliferation: DCs were incubated or not with the reported live bacterial strains for 1 h in medium without antibiotics, washed and incubated for 23 h in medium with antibiotics. Bacteria-treated DCs were washed and incubated with naïve CD4⁺CD45RA⁺ cells for 3 days, followed by a 16-hours pulse with 1 μCi [3H] thymidine (Amersham, Milan). ³H-thymidine incorporation is shown. Each symbol represents a different DC donor. Red lines represent mean values. *, p<0.05.

S. typh: S. typhimurium; L. plan: L. plantarum; L. par: L. paracasei.

B. Cytokine release: Bacteria-treated DCs were incubated with naïve CD4⁺CD45RA⁺ cells for 5 days (Ratio 1:10 DC:T cells). Cell culture supernatants were collected and cytokines measured by ELISA or CBA Flex set. Error bars: standard deviation on values obtained on 3 different donors. *, p<0.05; **, p<0.01.

FIG. 3. L. paracasei inhibits the release of inflammatory cytokines both directly and indirectly on DCs.

Each treatment is schematically reported below the graphs. Three situations were analyzed (a, b, c).

a. DCs were incubated or not with the reported live bacterial strains either separately (SL, S. typhimurium; LP, L. paracasei) or together (LP+SL) for 1 h in medium without antibiotics, washed and incubated for 23 h in medium with antibiotics.

b. Caco-2 cells were grown as monolayers in the upper chamber of a transwell filter. 24 h from achievement of a TER of 300 Ohm·cm² supernatants (sn Caco) were collected from the bottom chamber and used to pre-treat DCs for 24 h before bacterial incubation as in a.

c. Caco-2 cells were grown as monolayers in the upper chamber of a transwell filter and incubated with L. paracasei (5×10⁷ CFU/TW) upon the apical surface (top chamber). One hour after incubation, bacteria were washed out and medium was changed with one containing antibiotics. Culture supernatants (sn caco LP) were collected 24 hours later from the bottom chamber, filtered and used to pre-treat DCs for 24 h before bacterial incubation as in a.

24h after bacterial treatment of DCs cell culture supernatants were collected and cytokines analyzed by ELISA.

Error bars: standard deviations on values obtained on 3 different donors. *, p<0.05; **, p<0.01.

FIG. 4. An anti-inflammatory activity is found in the culture supernatant of L. paracasei.

DCs were incubated or not with the reported live bacterial strains either separately (SL, Salmonella; LP, L. paracasei) or together (LP+SL) or in the presence of culture supernatants of L. paracasei corresponding to the exponential growth of the same amount of CFU of bacteria used to treat the DCs. The culture supernatant (sn LP) was used either undiluted or diluted ⅕, 1/10, 1/100 that correspond to nearly 7%, 1.4%, 0.7%, and 0.07% volume/volume of tissue culture medium, respectively.

Cells were incubated with the different treatments for 1 h in medium without antibiotics, washed and incubated for 23 h in medium with antibiotics. Cytokine release was analyzed by ELISA. Error bars: standard deviations on values obtained on 3 different donors. *, p<0.05; **, p<0.01.

FIG. 5. L. paracasei (CNCM I-1390) inhibits the ability of DCs to activate T cells.

Three situations were analyzed (a, b, c) as in FIG. 4.

a. DCs were incubated or not with the reported live bacterial strains either separately (SL, Salmonella; LP, L. paracasei) or together (LP+SL) for 1 h in medium without antibiotics, washed and incubated for 23 h in medium with antibiotics.

b. Caco-2 cells were grown as monolayers in the upper chamber of a transwell filter. 24 h from achievement of a TER of 300 Ohm·cm² supernatants (sn Caco) were collected from the bottom chamber and used to pre-treat DCs for 24 h before bacterial incubation as in a).

c. Caco-2 cells were grown as monolayers in the upper chamber of a transwell filter and incubated with L. paracasei (5×10⁷ CFU/TW) upon the apical surface (top chamber). One hour after incubation, bacteria were washed out and medium was changed with one containing antibiotics. Culture supernatants (sn caco LP) were collected 24 hours later from the bottom chamber, filtered and used to pre-treat DCs for 24 h before bacterial incubation as in a.

Cells were then washed and incubated with naïve CD4+CD45RA+ cells for 5 days (Ratio 1:10 DC:T cells). Cell culture supernatants were collected and cytokines measured by ELISA or CBA Flex set.

Error bars: standard deviations on values obtained on 3 different donors. *, p<0.05; **, p<0.01.

FIG. 6. L. paracasei protects against DSS colitis.

Mice (n=6) were administered intragastrically (i.g.) once a day for 7 days with 200 μl PBS containing 10¹⁰ CFUs of bacteria or plain PBS as a control. Mice were then fed with 2% DSS dissolved in the drinking water for 5 days without bacteria, followed by 7 days of plain water and assessed over time for colitis development.

A. Body weight was measured at baseline and every day for the duration of the experiment. Weight change was calculated as percentage change in weight compared with baseline. L. plant.: L. plantarum; L. parac.: L. paracasei. Asterisks refer to statistical analysis of the groups LGG or L. plantarum versus DSS PBS positive control group. *, p<0.05; **, p<0.01; =, dead animals.

B. Disease activity index (DAI) was measured as reported in Materials and Methods. DAI at 4, 5, 7 days is shown per each group. The scatterplot shows a line at the mean of each group with error bars. Dashed lines identify one standard deviation above and below the group means. *, p<0.05; **, p<0.01.

FIG. 7. Serum levels of inflammatory and anti-inflammatory cytokines.

TNF-α (A) and IL-10 (B) serum levels were measured in normal rats (normal), rats with CCL₄ induced fibrosis (CCl₄) and rats with CCl₄ induced fibrosis treated with the probiotic (CCl₄+pr).

FIG. 8. Effect of probiotic treatment on the expression levels of mediators of the immune response.

A. Gel electrophoresis of PCR products from mouse liver with TNF-α, IL-10, IL-1β specific primers (GAPDH as internal control): differences in gene expression are observed between fibrotic rats (left side) and fibrotic rats treated with probiotic L. paracasei (right side).

B. Gel electrophoresis of PCR products from mouse liver with primers specific for mediators of the immune response: cytokines, Toll-like receptor and nitric oxide synthase isoforms. In particular: TGF-β, eNOS, iNOS, TRL4, TRL2 and GAPDH as internal control. Differences in gene expression between fibrotic rats (left side) and fibrotic rats treated with probiotic (right side) were observed

FIG. 9. Histological images of rat liver tissue

A. Rat liver in which liver fibrosis was induced with CCl₄.

B. Sirius Red staining on rat liver fibrotic tissue induced by CCl₄.

C. Sirius red staining on liver tissue of rats with fibrosis induced by CCl₄+L. paracasei.

### DETAILED DESCRIPTION OF THE INVENTION

According to a first embodiment, the present invention discloses an in vitro assay which allows to select probiotic strains useful in the therapy or, more preferably as a co-adjuvant therapy in IBD, i.e. bowel diseases with a strong inflammatory component and among these, namely ulcerous colitis or Crohn's disease.

In fact, as reviewed above, administration of probiotics is not always beneficial, and up to now and to the best of our knowledge, no predictive test for probiotics is available, with the exception of in vivo preclinical models (such as the Dextran Sulphate Sodium induced colitis in mouse). Therefore the present invention provides for the first time an assay in vitro for the selection of probiotic bacteria highly predictive of their activity in vivo.

This assay has furthermore confirmed the hypothesis that beneficial probiotics possess not only an intrinsically low inflammatory potential (i.e. do not stimulate the release of pro-inflammatory cytokines on their own) but also, and more importantly, that this combines with their most important ability to switch off the inflammatory response induced in immuno-competent cells by other bacteria or, in general by a strong bacterial stimulus. This finding is particularly important in the gut environment where the balance with intestinal microflora, variable in composition and characteristics, not ultimately depends on the modulation of the immune response of the host.

Specifically the assay provides that the probiotic strain to be tested is co-incubated with immunocompetent cells, preferably Dendritic Cell (DC) and with a strong bacterial antigen, such as preferably S. typhimurium or E. coli (selected strains). In some situations, commensal bacteria selected among: Clostridium, Bifidobacterium or enterobacteriacee further not excluding bacteroides, bifidobacterium, eubacterium, clostridium, peptococcus, peptostreptococcus, and ruminococcus, which are predominant in human beings, or in particular strains of Enterococcus, Klebsiella, and Proteus or even some Lactobacillus strains excluding paracasei strain CNCM 11390, may represent a strong bacterial stimulus. Isolated bacterial protein may also represent a strong bacterial antigen (i.e. LPS).

Putative probiotics may be also pre-incubated on dendritic cells and placed afterwards in contact with the strong bacterial stimulus.

Specifically, the assay provides that after co-incubation at least the level of two or preferably three cytokines is measured and compared to their levels in the absence of the probiotic strain or its supernatant. In particular the assay provides that in the presence of a beneficial probiotic strain, or its supernatant alone, the IL-12p70 levels, and preferably also the TNF-α levels decrease with respect to the level measured in its absence. IL-10 production is also measured and its levels compared to those measured in the absence of the probiotic strain.

The comparison between the levels produced in the absence or in the presence of the probiotic strain provides a statistically relevant decrease in IL-12p70 (where by IL-12p70 is meant the bioactive form of IL-12, combining a p35 and a p40 subunit) and preferably also TNF-α levels, and a substantially unaltered level of IL-10 production upon co-incubating conditions.

Furthermore, as said above, the test provides that the DC cytokine activation pattern upon co-incubation of DC and the sole probiotic, or its supernatant, is almost unaltered with respect to the pattern of unstimulated DC, i.e. that, in summary, the probiotic bacteria is endowed with intrinsically low DC activating properties.

It follows that according to a preferred embodiment, the assay provides a test where, in parallel with the above, a probiotic strain, or the supernatant thereof, is co-incubated with dendritic cells and the levels of IL12p70, TNF-α and IL-10 are measured, in the absence of any pro-inflammatory signal. The selection criteria for the probiotic strain is in this case a cytokine expression pattern where IL12p70 is reduced, TNF-α is reduced and IL-10 is unaltered when preferably measured after 24 hrs co-incubation.

DC are preferably derived from human peripheral blood monocytes, purified for example by positive selection with anti-CD14 antibodies coupled to magnetic beads or by FACS analysis. CD14⁺ cells are preferably incubated for 6 days in complete medium containing granulocyte-macrophage colony-stimulating factor (GM-CSF, 50 ng/mL; Peprotech) and interleukin-4 (IL-4, 20 ng/mL; Peprotech, Milan, Italy) in order to obtain immature MoDCs.

MoDC are then plated under culturing conditions known in the art, usually represented by a synthetic culture medium such as: RPMI 1640 comprising FCS or growth factors.

Cytokine measurement is preferably carried out by immunoassay with commercial antibodies or commercial assay, preferably ELISA tests. Cytokine measurement can be also carried out according to known methods, for example by functional assays, such as the induction of immune system function (i.e. Th1 activation) or radioimmunoassays.

By the assay of the present invention, three different probiotic strains: L. plantarum, LGG and L. paracasei (Collection Nationale de cultures de Microorganismes, Institute Pasteur, accession number CNCM I-1390, described in EP 0760848) have been shown to possess a dramatical difference in their ability to activate DCs. In fact, only co-incubation of DCs with S. typhimurium as the bacterial antigen and L. paracasei (CNCM 1390) among the three probiotics, strongly inhibits the release of IL-12p70, sparing the release of IL-10, blocking in turn Th1 T cells induction by DC.

This could not be foreseen unless by testing their activity, directly, in vivo.

According to an embodiment of the assay, co-incubation of the probiotic strain or its supernatant on DCs can be carried out at the same or different times. For example, DC can be pre-incubated at first with the putative probiotic strain for at least 30′, preferably 1 hour, before adding the strong bacterial stimulus. Incubation of the probiotic strain on DC is carried out in the absence of any antibiotics.

1 hour co-incubation of DCs with the putative probiotic strain or supernatant thereof, in the presence or absence of the strong bacterial antigen is usually enough to observe any effect on DCs.

A strong bacterial antigen, such as S. typhimurium or antigens thereof, may be represented by particular strains of E. coli or some commensal bacteria and antigens thereof. Strong DCs stimuli may be represented also by inflammatory stimuli, such as LPS.

However, for measuring DC cytokines secretion pattern, at least a further incubation of 8 hrs, preferably 10, or even more preferably 15 or 20 hours, after would be needed, or even more preferably a time-course response of culture in a range from 8 to 24 hours.

As an experimental confirmation to the hypothesis that the specific cytokine pattern observed with beneficial probiotics correlates and is relevant to the downstream lack of activation of Th1 cell, has been provided by further co-incubating the DC cells, already treated as described above, with allogeneic naive T-lymphocytes CD4+CD45RA+. Incubation of DCs with a beneficial probiotic, preferably L. paracasei impairs their ability to secrete cytokines usually correlated with Th1 polarization (IFN-γ, TNF-α) and, in turn, their activation.

According to this further aspect of the assay, L. plantarum has been found to be more similar to S. typhimurium in terms of DC activation and T cell polarization than the other two strains. L. paracasei is very poor in activating DCs and in inducing cytokine production (both inflammatory and non-inflammatory), while LGG displayed an intermediate phenotype between paracasei and S. typhimurium. Also when the capacity of the different Lactobacilli to activate epithelial cells (EC) was tested, L. paracasei was less inflammatory as it induced an increased release of TGF-β and TSLP. Both TGF-β and TSLP play an important role in the human system to drive the differentiation of non-inflammatory DCs (Iliev et al. Gut 2009). The effect of L. paracasei was very dramatic as it affected the ability of DCs to respond to S. typhimurium both directly (when co-incubated with Salmonella) and indirectly after treatment of DCs with supernatants from ECs having encountered L. paracasei from their luminal side.

The incubation of DCs with supernatants of ECs treated with L. paracasei also affects the release of IL-12p70 and has an even more dramatic effect on T cells as it strongly inhibits their activation, particularly in the development of Th1 T cells.

Hence, the present invention provides that the cytokine pattern observed in the condition summarized above, strongly correlates with the inhibition of T cell activation in response to strong immunogens like S. typhimurium. The effect of L. paracasei on DC activation is not limited to S. typhimurium but also applies to other immunogenic Lactobacilli suggesting that once in the intestine L. paracasei may broadly inhibit the activation of the immune system in response to immunogenic commensal bacteria by acting both directly on DCs and indirectly on ECs.

The observed cytokine pattern and selection criteria of the method of the present invention for probiotic strains, in the presence of a strong inflammatory stimulus, strongly correlates with protection from colitis in a murine model of DSS (dextran sulphate sodium) induced acute colitis. In fact, among the probotic strains tested, only L. paracasei, administered to mice before induction of DSS colitis, exherted a protective effect.

Thus, results obtained by the test of the present invention, have been confirmed in the preclinical model, showing the feasibility of the method. This is with the more surprising in view of the reported strong ability of L. casei, a close Lactobacillus strain, to induce maximal levels of both IL-12 and TNF-α (Christensen H. R., J. Immunol (2002) 168:171-178). Probiotics belonging to the Lactobacillus genus for which this assay is proposed, are preferably specific strains within the genera selected in the group consisting of: Lactobacillus johnsonii, Lactobacillus reuterii, Lactobacillus paracasei, Lactobacillus casei, Lactobacillus animalis, Lactobacillus ruminis, Lactobacillus acidophilus, Lactobacillus rhamnosus, Lactobacillus fermentum, and Lactobacillus delbrueckii subsp. Lactis.

Even more preferably, lactobacillus strains are those selected from the group consisting of Lactobacillus johnsonii La1 NCC 2461 (=CNCM I-2116), Lactobacillus reuterii strains 4000 and 4020 (from BioGaia Biologics Inc., Raleigh, N.C.), Lactobacillus paracasei strain CNCM I-1390 (Bracco internal code: 21060), Lactobacillus casei strain Shirota, Lactobacillus acidophilus strain CNCM I-1447, Lactobacillus acidophilus Lat 11/83, Lactobacillus acidophilus NCC 2463 (=CNCM I-2623), Lactobacillus rhamnosus GG (ATCC 53103), Lactobacillus rhamnosus 271 (DSMZ 6594) and Lactobacillus rhamnosus VTT E-800.

Probiotic belonging to the genera of bifidobacterium are preferably selected in the group consisting of: Bifidobacterium spp., Bifidobacterium bifidum, Bifidobacterium longum, Bifidobacterium pseudolongum, Bifidobacterium infantis, Bifidobacterium adolescentis, and Bifidobacterium lactis.

Even more preferably, bifidobacterium strains are selected from the group consisting of: Bifidobacterium bifidum NCC 189 (=CNCM I-2333), Bifidobacterium adolescentis NCC 251 (=CNCM I-2168), Bifidobacterium lactis (ATCC 27536), Bifidobacterium breve CNCM I-1226, Bifidobacterium infantis CNCM I-1227, and Bifidobacterium longum CNCM I-1228.

Indeed by using the test developed, at least two Lactobacillus strains (rhamnosus GG and plantarum) have been predicted to worsen rather than to protect against colitis, while only Lactobacillus paracasei CNCM I-1390 was protective in this model, providing further evidence that it's very important to fully investigate the activity of each probiotic before proposing them for any human use.

Accordingly, this test is advantageously used to avoid indiscriminate use of probiotics, thus allowing the preparation of probiotic compositions beneficial for conditions of the gastrointestinal tract, in particular those with a strong inflammatory component such as ulcerative colitis, Crohn's disease, Collagenous colitis, Lymphocytic colitis, Ischaemic colitis, Diversion colitis, Behçet's syndrome, Indeterminate colitis.

As said above, the method of the present invention, has allowed the evaluation of the potential of dendritic cells activation (and downstream immune system activation, in particular Th1 activation) of three Lactobacilli strains (plantarum, rhamnosus GG and paracasei CNCM I-1390, Bracco internal code: 21060) either directly or indirectly even via their conditioned medium. The activity of the three probiotics is very different, being Lactobacillus paracasei CNCM 1-1390 the only strains able to inhibit the inflammatory potential of pathogenic S. typhimurium in vitro thus demonstrating its immunomodulatory properties. These results, surprisingly, strongly correlate with the ability of such a strain to protect animals in vivo, against experimental colitis.

Conversely, the in vitro systems of the present invention, could also predict the immunostimulatory properties of L. plantarum and LGG and may reasonably explain why LGG is so potent in preventing nosocomial rotavirus dependent diarrhea in infants, while it may even be detrimental in IBD or related diseases, with the more pointing to the usefulness of the assay.

Selected probiotics can thus be used in the treatment of inflammatory bowel diseases, in particular Crohn's or ulcerative colitis, or as a co-adjuvant (additive) therapy in the same diseases, together with drugs commonly used in these pathologies, such as mesalamine, TNF inhibitors or steroids, depending on the severity of the disease.

Once the probiotic is selected as “beneficial”, the present invention provides for its use, alternatively or in addition to the above use, as a maintenance therapy for the same diseases, i.e. to delay or avoid their recurrence.

A particularly preferred selected probiotic is Lactobacillus paracasei (CNCM I-1390 Bracco internal code: 21060) or its supernatant (also “conditioned” medium) for use in the treatment of inflammatory bowel diseases, in particular Crohn's or ulcerative colitis, or as a co-adjuvant therapy in the same diseases, together with drugs commonly used in the treatment of these pathologies, such as mesalamine, TNF inhibitors or steroids, depending on the severity of the disease.

Given the biologic activity of Lactobacillus paracasei strain tested and supernatant thereof on dendritic cells in switching off their pro-inflammatory potential, these strains are thus proposed, also, as a maintenance therapy for IBD, intestinal diseases with an important inflammatory component, namely colitis or ulcerative colitis, namely to delay or avoid recurrence of such diseases, taken alone or in combination with drugs such as, i.e. mesalamine, biological agents as TNF inhibitors or steroids

According to a further aspect of the invention, the method comprises the selection of a probiotic strain, according to the assay mentioned above, and its formulation into a composition for oral administration such as in compositions generally intended for gastrointestinal use, to be preferably administered as a drink, a capsule, an infant formula or a dairy product.

To this extent, the selected bacterial strains may be suitably employed so that the amount of bacteria or its supernatant, available for administration, corresponds to about 10³ to about 10¹⁴ CFU per day, preferably from about 10⁷ to about 10¹² CFU per day, and even more preferably from about 10⁹ to about 10¹² CFU per day. When only the supernatant is formulated into the composition, the corresponding amount of medium conditioned by the above quantity of bacteria is to be used.

For oral formulation any proper form for oral assumption is to be intended, such as, among others, a milk drink, a yoghurt-similar milk product, a cheese, an ice-cream, a fermented cereal-based product, a milk-based powder, an infant formula, a tablet, a capsule, a liquid suspension, a dried oral grit or powder, a wet oral paste or jelly, a grit or powder for dry tube feeding or a fluid for wet tube feeding.

Alternatively, the drink may be prepared before use from a dissolvable capsule containing the active ingredients.

Preferably, the drink may be prepared before use by reconstituting a dry powder containing the lyophilized bacteria and their supernatant or, alternatively, by reconstituting a dry powder containing the lyophilized bacteria and their supernatant with an aqueous solution.

The dry powder is preferably packaged in such a way that the stability of the solid may be retained along the time, such as for instance, into airtight and light-tight sachets, under air or nitrogen, under a noble gas or under vacuum.

As far as the capsules are concerned, they may be properly manufactured according to conventional methods.

From all of the above, it is clear to the skilled person that the compositions of the invention may further comprise any additional excipients among those commonly employed in pharmaceutical formulations, in order, for instance, to stabilize the compositions themselves, or to render them easily dispersible or to give them an agreeable taste.

Among said excipients inulin, fructose, starch, xylo-oligosaccharides, silicon oxide, buffering agents as well as flavors, are suitable examples.

Furthermore, optional active ingredients may be also present in the compositions of the invention such as, for instance, vitamins, amino acids, polypeptides and the like.

An example of an optional active ingredient may be represented by glutamine which may help intestinal cells to defend themselves under stress conditions due to pathogenic organisms (U.S. Pat. No. 6,007,808).

Alanyl-glutamine as well as a variety of vitamins may also represent additional optional ingredients within the compositions.

The presence of transition metals should be preferably avoided so to not impair the binding and/or sequestration of the naturally occurring iron ions by the chelator. However, by considering that the preferred chelators according to the invention bind iron ions much stronger than other physiological transition-metal ions, for instance zinc or copper, the presence of these latter substantially does not affect the efficacy of the present compositions.

According to a further embodiment of the present invention, Lactobacillus paracasei strain CNCM I-1390, or supernatant thereof, is proposed for use in the treatment of chronic liver diseases, in particular fibrosis due to toxic or metabolic causes such as chronic alcohol abuse, continuous hepatotoxic drug assumption, obesity, diabetes and dyslipidemic disorders. Compositions comprising the selected Lactobacilli, or their supernatants are used as such or as a co-adjuvant therapy, together with other treatments (additive therapy), depending on the severity of the disease. Alternatively, they may be used in the prevention of the same diseases, or in the maintenance of the disease in a steady state, together with a suitable dietary regimen. As a matter of fact, no useful therapy is presently available for liver fibrosis.

The results obtained in a model of experimentally induced liver fibrosis in rat, consistent with the cytokine production and expression pattern observed in vitro, are in line with the hypothesis that Lactobacillus paracasei has actually an effect on modulation of immune pathways and on the fibrogenic mechanisms in the liver. In fact, administration of L. paracasei or its supernatant has been shown to limit the deposition of fibrotic fibers (collagen) into the organ, thus showing an important role in the prevention of the pathology.

The invention further relates to a method for the treatment of inflammatory bowel diseases in a mammal comprising administering to a mammal in need thereof an effective amount of Lactobacilli paracasei (CNCM I-1390) or its supernatant, in a oral composition thereof. Preferably said IBD is a ulcerative colitis or a Crohn's disease.

According to a further embodiment of the present invention, a method for the treatment of a hepatic chronic disease in a mammal comprising administering to a mammal in need thereof on effective amount of a Lactobacillus paracasei strain CNCM I-1390, or supernatants thereof in a oral composition, is herein provided. By liver chronic diseases we refer in particular to fibrosis, due to toxic or metabolic causes such as chronic alcohol abuse, continuous hepatotoxic drug assumption, obesity, diabetes and dyslipidemic disorders.

By this assay, able to recognize immunomodulatory and immunostimulatory strains, it has been confirmed that not all Lactobacilli can protect against colitis; rather, immunostimulatory strains like plantarum or LGG may even be detrimental; thus restraining their use, for example, to other pathologies than inflammatory bowel disorders.

By using the assay of the present invention, it has been found that, surprisingly, not only the probiotic bacteria Lactobacillus casei CNCM I-1390 (Bracco internal code 21060), is in particular endowed with the ability to switch off the immunomodulatory activity in DC upon co-stimulation with a strong bacterial stimuli, but, also, that this property extends to the supernatant alone, depleted of bacterial cells.

Therefore, according to a further embodiment, the invention provides for the conditioned supernatant of lactobacillus, preferably Lactobacillus paracasei CNCM I-1390 as a therapy and/or a co-adjuvant therapy, beneficial for treating a condition selected from the group consisting of: inflammatory bowel disease, ulcerative colitis and chronic liver condition such as steatohepatitis and liver fibrosis, through its activity on dendritic cells by the above identified mechanism.

Liver fibrosis may have toxic or metabolic causes, among the former, i.e chronic alcohol abuse and continuous hepato-toxic drug assumption; among the latter, i.e. obesity, diabetes or dyslipidemic disorders.

When the bacteria supernatant, otherwise called conditioned medium, is used, this is prepared according to methods known in the art. Culturing conditions for the strains above mentioned have been widely described, i.e. in EP760848.

A satisfactory bacterial growth for the purpose of the present embodiment, is achieved by culturing said strains, preferably in anaerobiosis, in MRS broth (Oxoid or Biokar) or similar medium, up to an exponential growth phase, generally achieved after restarting an overnight culture at 1:50 at 37° C. for a time of at least 1 hour or preferably at least 2 hours in fresh medium. Preferably, the selected strain of Lactobacillus paracasei (CNCM 1390) are grown overnight anaerobically at 37° C. in MRS broth (Biokar Diagnostic) without shaking. Bacterial cultures for the preparation of the supernatant may be restarted in fresh culture medium using dilutions comprised from 1:5 to 1:100 up to an OD 600 of about 0.6, roughly corresponding to an exponential growth phase (comprised from 0.9 to about 1.4×10⁹ CFU, or even more preferably comprised from 1.1 to 1.3, or 1.2×10⁹ CFU/ml). Bacterial cultures may be plated to count effective CFUs. L. paracasei supernatant for the oral use formulations and/or for the DC assay may be obtained after centrifugation of the equivalent amount of CFUs of an exponentially growing bacteria. In the DC assay, L. paracasei supernatant may be used either undiluted, or diluted from about 1:5 to about 1:100.

Depletion of bacterial cells from the culture medium is conveniently effected by centrifugation, filtration on suitable membranes or chromatography, or even by simple sedimentation of the bacterial pellet.

The skilled man, would suitably select alternative means for cell depletion, keeping in mind that safety of the product is not at issue in the present case, since Lactobacilli are eubiotic bacteria and are safely used in humans. The issue is rather the selection of the most suitable method according to further storage needs or down-stream processes of the supernatant, such as concentration, lyophilisation, fractionation on the basis of molecular size or biochemical properties or, even, in an alternative embodiment of the present invention, for recycling bacterial cells for the production in continuous of the conditioned medium.

The use of the supernatant instead of the culture of live bacteria offers several advantages, because the supernatant can be easily concentrated and formulated in a suitable dosage and even more easily stored than the alive bacteria.

### Experimental Part

### Materials and Methods

### Mice and Bacterial Strains

C57/BL6 mice were purchased from Charles River laboratories. All mice were maintained in microisolator cages in a specific pathogen-free animal facility. All experiments were performed in accordance with the guidelines established in the Principles of Laboratory Animal Care (directive 86/609/EEC) and approved by the Italian Ministry of Health.

S. typhimurium strain SL1344 was provided by G. Dougan (The Wellcome Trust Sanger Institute, UK) and grown in LB medium. Lactobacilli strains were: L. plantarum, NCIMB8826 WT; L. paracasei (CNCM I-1390, internal code B21060, Bracco); L. rhamnosus GG (Dicoflor 30, Dicofarm). All Lactobacilli were grown overnight anaerobically at 37° C. in MRS broth (Biokar Diagnostic) without shaking. Bacteria were restarted at a 1:50 dilution and grown to an OD 600=0.6 when the growth is exponential. Bacterial cultures were plated to count effective CFUs. L. paracasei supernatant was obtained after centrifugation of the equivalent amount of CFUs of exponential phase bacteria used for DC treatment. L. paracasei supernatant was used either undiluted, or diluted 1:5, 1:10 or 1:100 corresponding to nearly 7%, 1.4%, 0.7%, and 0.07% volume/volume of tissue culture medium, respectively.

### Cells and Reagents

DCs were derived from human peripheral blood monocytes selected with anti-CD14 antibodies coupled to magnetic beads (Miltenyi, Bologna, Italy) [29]. CD14+ cells were incubated for 6 days in complete medium containing granulocyte-macrophage colony-stimulating factor (GM-CSF, 50 ng/mL; Peprotech) and interleukin-4 (20 ng/mL; Peprotech, Milan, Italy) in order to obtain immature MoDCs.

### Bacterial Treatments and Assessment of MoDC Viability.

MoDCs were incubated for 1 h with live logarithmic-phase Lactobacilli (L. plantarum, L. paracasei and LGG) or with Salmonella typhimurium in medium without antibiotics at a 10:1 (bacteria:DC) ratio. Cells were extensively washed and the medium was changed to one containing gentamycin (100 μg/ml). Cells were tested 24 hours later for viability after double staining with FITC-conjugated Annexin V (BD PharMingen, San Diego, Calif.) and 1.25 μg/ml propidium iodide (Sigma Chemical Co.), and analyzed by flow cytometry. Annexin V/propidium iodide double negative cells are indicative of viable cells.

### Epithelial Cell Monolayers

Caco-2 cells were seeded in the upper chamber of a transwell filter (Costar 3 μm diameter of pores) for 7-10 days until a trans-epithelial resistance (TER) of 300 Ohm·cm² was achieved.

Epithelial cell monolayers were incubated with bacteria (5×10⁷ CFU/TW) upon the apical surface (top chamber). One hour after incubation, bacteria were washed out and medium was changed with one containing antibiotics (gentamycin 100 μg/mL). Culture supernatants were collected 24 hours later from the bottom chamber (facing the basolateral membrane), filtered through a 0.2 μm filter (Nalgene) and used to activate MoDCs.

MoDCs were incubated for 24 hours in culture supernatant (1:2) and then treated or not with bacteria (10:1 bacteria to DC) for 1 h in medium without antibiotics. Subsequently bacteria were washed out and cells were left in culture for an additional 23 h in medium containing gentamycin 100 μg/mL. Analysis of cytokines released by epithelial cells or DCs was carried out by testing culture supernatants.

### MoDC T-Cell Co-Cultures

MoDCs were collected after 24 hours of incubation with the different stimuli and then incubated with allogeneic CD4⁺CD45RA⁺ purified T cells (Miltenyi) in 48-well plates (at a ratio of 10 T cells to 1 DC). To measure T cell proliferation: MoDC-T cell were co-cultured for 72 hours, followed by a 16-hours pulse with 1 μCi [³H] thymidine (Amersham, Milan). Cell-associated radioactivity was detected after Cell harvesting (TomTec) on filter mats using a Betaplate Counter (MicroBeta TriLux, PerkinElmer).

To measure cytokine release: after 5 days of MoDC-T cell co-culture, supernatants were collected and directly analyzed for cytokine measurements.

### DSS Colitis

6 mice per group were administered intra gastrically (i.g.) once a day for 7 days with 200 μl PBS containing 10¹⁰ CFUs of bacteria grown as above. Control mice were administered with plain PBS. Mice were then fed with 2% DSS dissolved in the drinking water for 5 days without probiotics, followed by 7 days of plain water and analyzed over time for colitis development. Mice were weighed every day and feces were collected to measure consistency and the presence of blood by HEMOCCULT (BeckmanCoulter, Inc). At day 13 after DSS treatment mice were sacrificed.

### Assessment of Disease Activity (DAI).

Body weight was assessed at baseline and every day for the duration of the experiment. Weight change was calculated as percentage change in weight compared with baseline. Animals were monitored clinically for rectal bleeding, diarrhea and general signs of morbidity, including hunched posture and failure to groom. Disease activity index (DAI) is the combined score of weight loss, stool consistency, and bleeding. Scores were defined as follows: body weight loss, 0, no loss; 1, 5%-10%; 2, 10%-15%; 3, 15%-20%; 4, 20%; stool consistency, 0, normal; 2, loose stool; 4, diarrhea; bleeding, 0, no blood; 2, presence of bleeding; and 4, gross bleeding.

### Cytokine Measurements

IL-6, IL-2, IL-12p40, IL-17, IL-12p70, IL-10, IFN-γ, TNF-α and TGF-β concentrations were determined by commercially available ELISA (R&D systems) or Cytokine bead array (Becton Dickinson). Optical densities were measured on a Bio-Rad Dynatech Laboratories ELISA reader at a wavelength of 450 nm (Hercules, Calif., USA). CBA-associated Cytofluorimetry was measured by FACS array (Becton Dickinson). Limit of detection of cytokines by CBA<10 pg/ml (for all of them) and by R&D systems TSLP<5 pg/ml, TGF-β<30 pg/ml, IL-8<30 pg/ml.

### Statistical Analysis.

Student's paired t test was used to determine the statistical significance of the data. Significance was defined as *, p<0.05; **, p<0.01; ***, p<0.001 (two-tailed test and two-sample equal variance parameters). Statistic calculations were performed by JMP 7 software (SAS Cary).

### Liver Fibrosis

Induction of liver damage was carried out according to well known procedures Von Montfort C. et al. Am J Physiol Gastrointest. Liver Physiol 2010 May;298(5):G657-66). Wistar male rats (weight 220-250 g), after one week of acclimation, were divided into three groups of ten rats each, as follows:

1. Normal rats,

2. Hepatic fibrosis induced rats,

3. Hepatic fibrosis Induced rats treated with a symbiotic composition containing Lactobacillus paracasei (21060 Bracco).

To induce the hepatic fibrosis each rat of the group 2 has received a subcutaneous injection of carbon tetrachloride (CCl₄) 50% diluted in olive oil. The carbon tetrachloride injection was done twice a week for seven weeks. The same protocol was used to induce hepatic fibrosis in rats of group 3 and they were treated with 1 ml of probiotic solution (200 mg/kg daily) by oral administration.

Blood, feces, urine basal collections were made before proceeding to the induction of liver injury and before starting the probiotic treatment. At the end of the 7 weeks treatment, feces and urine samples were re-collected; then the rats in each group were sacrificed to collect blood from the carotid artery and remove the liver.

The collected samples were analyzed to evaluate the following parameters:
intestinal permeability
intestinal flora composition
biochemical parameters of the liver damage
serum inflammation indexes
gene expression of TNF-α, IL-10, IL-1β6, TLR2, TLR4, eNOS and iNOS
determination of the fibrosis index by histological analysis.

### Intestinal Permeability

Gut permeability is an index of intestinal barrier functionality. It represents the passive passage through the intestinal epithelium of water and small inert soluble molecules through intercellular spaces. The measurement of intestinal permeability can be obtained by oral administration of molecular probes (sugars) and measurement of their urine amount within a minimum time of 5 hours. The probes we used are saccharose (MW 342.3 Da), lactulose (MW 342.3 Da) and mannitol (MW 182.17 Da).

Before and after 7 weeks of treatment with CCl4 rats in all three groups were tested for intestinal permeability as described in (Meddings J, Gibbons I; Gastroenterology 1998; 114: 83-92). The volume of urine, the composition within 24 hours was analyzed by HPLC coupled with pulsed amperometry (HPAE-PAD) based on a calibration curve by analyzing the area under the peaks.

### Measure of Biochemical Parameters of Liver Injury.

Blood samples taken before and after treatment were centrifuged at 3000 rpm for 10′ at room temperature to separate the serum. Serum samples were analyzed to determinate the levels of AST (alanine amminotransferase), ALT (aspartate amminotransferase), γ-GT (γ-glutamminiltransferase, ALP (alkaline phosphatase) and total bilirubin by colorimetric well standardized routine kits.

### Determination of Serum Biomarkers of Inflammation (TNF-α and IL-10)

Serum levels of mouse/rat TNFα and mouse/rat IL-10 were determined by quantitative ELISA-based test and immunoassay technique according to the instruction of the kit currently on the market (Quantikine ELISA-R&D Systems, Minneapolis, USA).

Gene Expression of TNF, IL-10, IL-1β, TLR2, TLR4, eNOS and iNOS by RT-PCR

The extraction of RNA from liver tissue was carried out according to the method of guanidine isothiocyanate (TRIZOL® methods).

Analysis of the mRNA levels of: TNF, IL-10 and IL-1β, TLR2, TLR4, eNOS and iNOS genes was performed by RT-PCR (Semiquantitative RT-PCR) according to known techniques with primers enlisted in Table 1.

TABLE 1
Primers used in the semiquantitative RT-PCR
experiments.

| GENE | | | |
|---|---|---|---|
| PRIMER | | SEQUENCE | |
| TNFα | For | 5′-AAATGGGCTCCCTCTCATCA-3′ | SEQ ID NO: 1 |
| | Rev | 5′-TCCTTAGGGCAAGGGCTCTT-3′ | SEQ ID NO: 2 |
| IL-10 | For | 5′-TGGCTCAGCACTGCTATGTTG-3′ | SEQ ID NO: 3 |
| | Rev | 5′-TCCAGAGGGTCTTCAGCTTCTC-3′ | SEQ ID NO: 4 |
| IL-1β | For | 5′-ACTTGGGCTGTCCAGATGAGA-3′ | SEQ ID NO: 5 |
| | Rev | 5′-GCCTGCAGTGCAGCTGTCTA-3′ | SEQ ID NO: 6 |
| TLR4 | For | 5′-CGCATAGAGACATCCAAAGG-3′ | SEQ ID NO: 7 |
| | Rev | 5′-TTCTCACCCAGTCCTCATTC-3′ | SEQ ID NO: 8 |
| TLR2 | For | 5′-CCCTTGACATCAGCAAGAAC-3′ | SEQ ID NO: 9 |
| | Rev | 5′-ACAGGAGTTCACAGGAGCAG-3′ | SEQ ID NO: 10 |
| eNOS | For | 5′-CTCAGGTTCTGTGTGTTTGG-3′ | SEQ ID NO: 11 |
| | Rev | 5′-GGATTTGCTGCTCTGTAGGT-3′ | SEQ ID NO: 12 |
| iNOS | For | 5′-AGCGAGTTGTGGATTGTTCT-3′ | SEQ ID NO: 13 |
| | Rev | 5′-CTTCGGGCTTCAGGTTATT-3′ | SEQ ID NO: 14 |
| GAPDH | For | 5′-GTCATACCAGGAAATGAGCT-3′ | SEQ ID NO: 15 |
| | Rev | 5′-GCCAAAAGGGTCATCATCTC-3′ | SEQ ID NO: 16 |

The amplification reaction was performed with an apparatus for PCR (Gene Amp 9700 Perkin Elmer) with the following protocol: the first step of the amplification reaction requires the DNA denaturation at 94° C. for 3 minutes. This was followed by 29 to 35 cycles comprising:
DNA denaturation at 94° C. for one minute
Annealing: one minute
Amplicon elongation at 72° C. for one minute.

In each RT-PCR experiment negative controls and normalization of mRNA levels with GAPDH gene (internal control: housekeeping gene) were carried out.

### Histological Analysis: Sirius Red Staining.

This staining uses Sirius Red particular affinity to the collagen fibers provide a useful parameter for evaluating the degree of fibrosis in liver biopsies. The selectivity of this compound against collagen proteins has allowed a computer-assisted quantitative study.

The Sirius Red staining was carried out by Fixation in 10% formalin on 4 μm sections with Solution A (Sirius Red F3B, gr. 0.1, picric acid sat. sol 100 ml)

Briefly, liver sections were paraffined, brought into distilled water and then stored in dark at room temperature in solution A for 15 minutes. A quick dehydration of the sections was made in three changes of absolute ethyl alcohol and then they were cleared by three xylene changes.

### Morphometric Analysis

By using a computer connected to a microscope equipped with camera (IA Leica Quantimet Q500 IW) is possible to do many different morphometric measurements on digital images acquired (image analysis).

### Image Processing and Analysis of Liver Fibrosis

Images of histological slides were processed through Leica Quantiment 500 IW image analysis system, and computer operations were performed using Windows with Leica QUIPS software (Quantitative Interactive Programming System).

For each section, 10 fields were examined through acquisition, digitization and analysis. Measurement was performed automatically by the system, which provided the percentage of collagen fibers in relation to stromal tissue and total area.

### Statistical Analysis

All the analysis results obtained were expressed as average±SD (standard deviation); comparisons between averages was carried out by the “T student test” for paired data. P values <0.05 were considered statistically significant.

### Example 1

### Phenotypic Activation of DCs and Cytokine Production

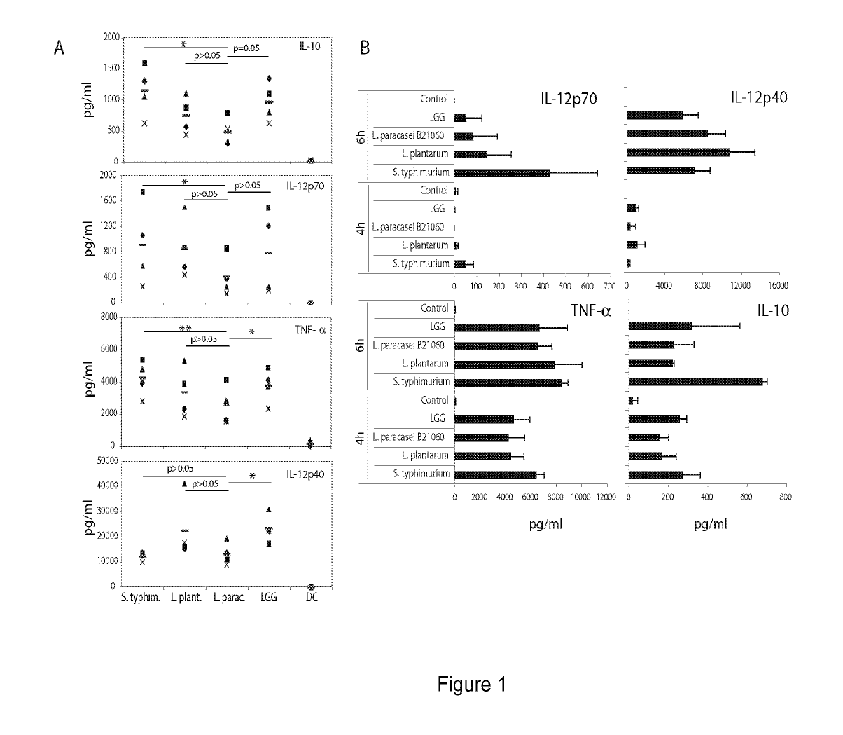

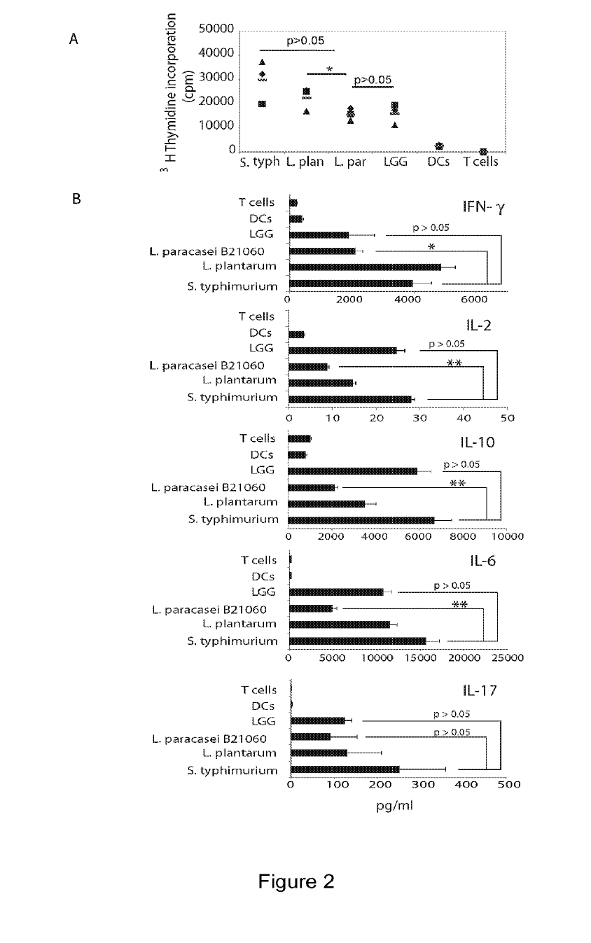

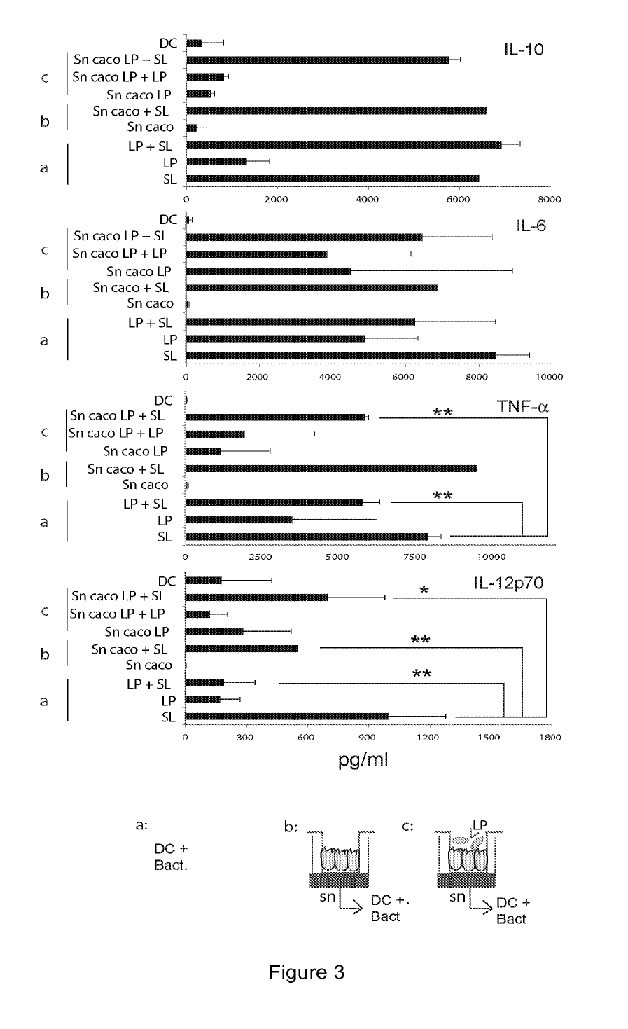

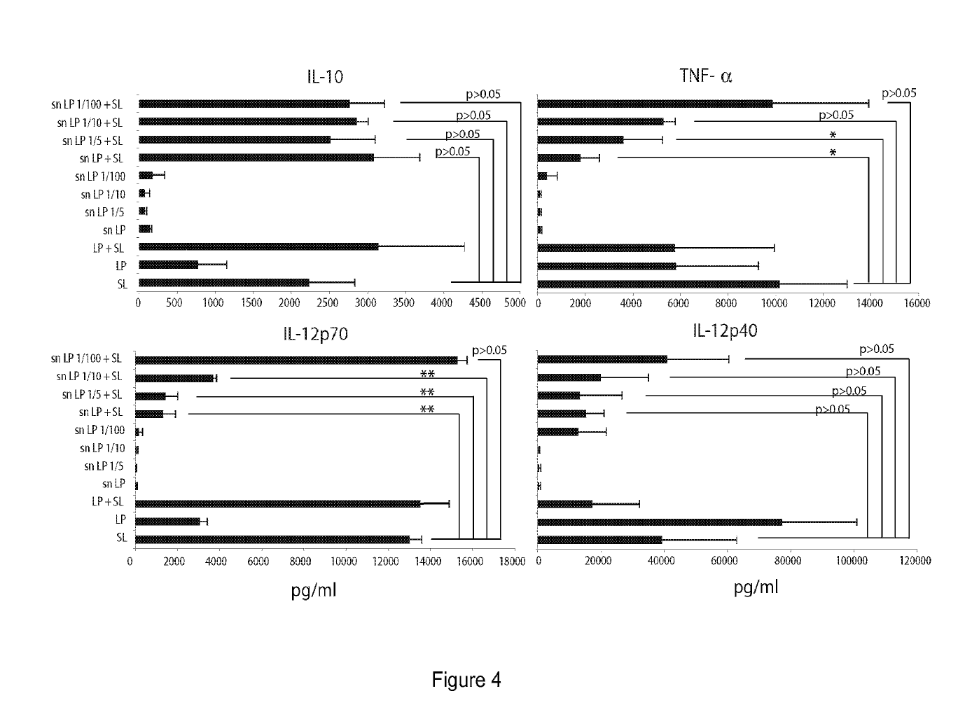

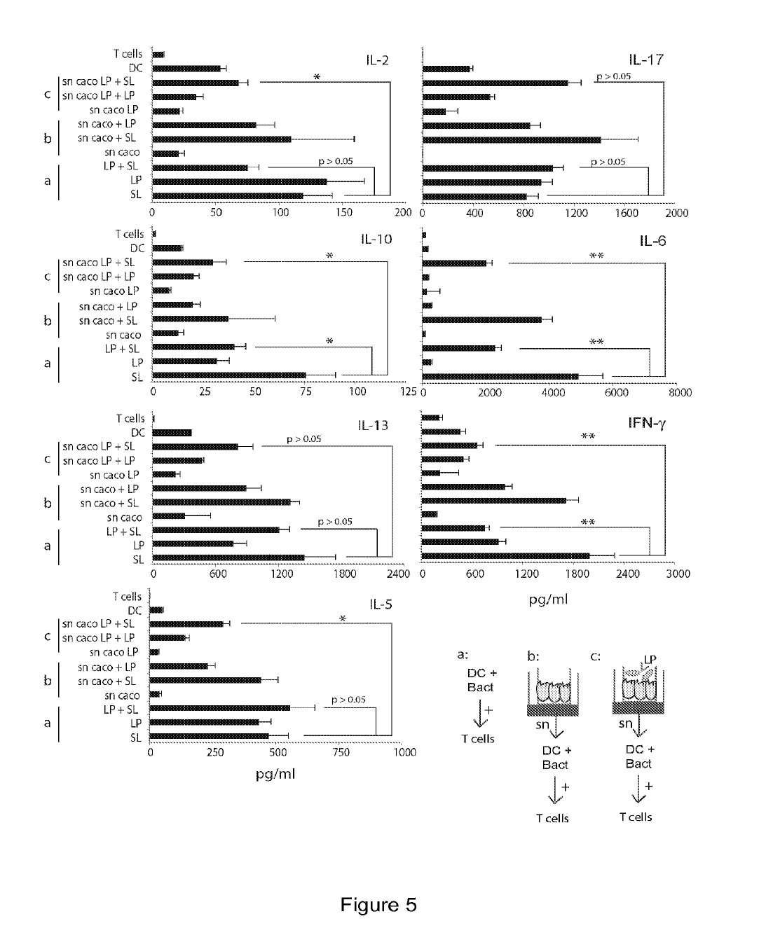

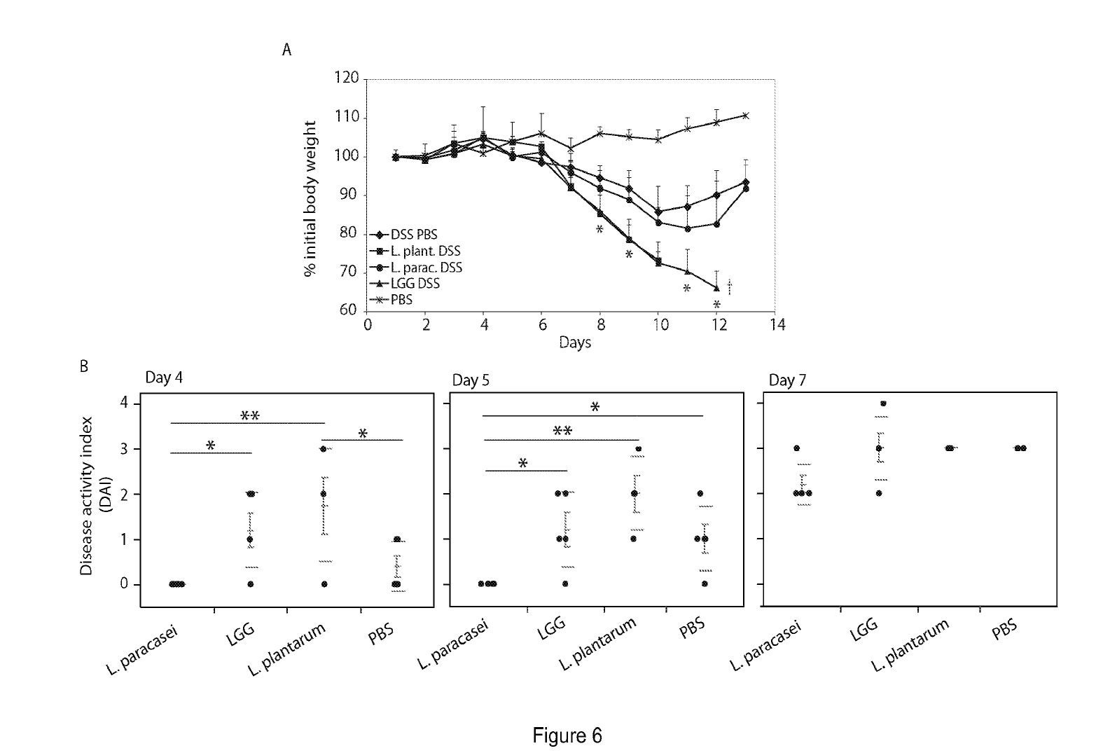

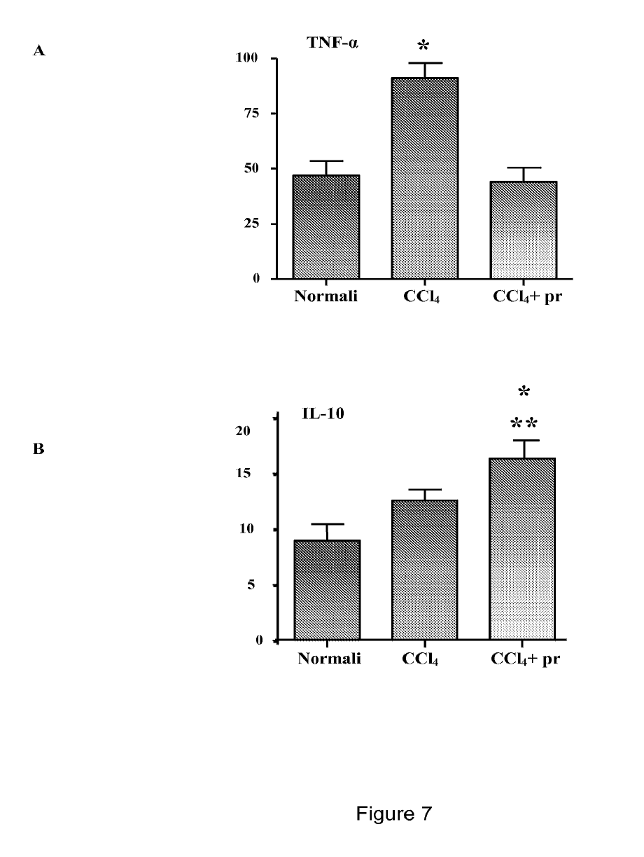

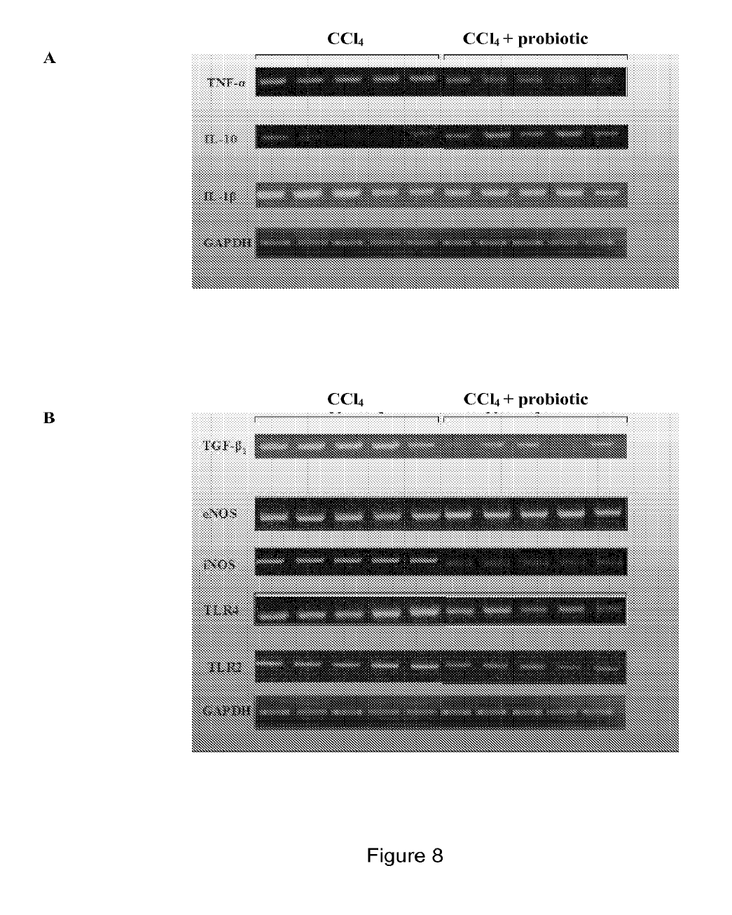

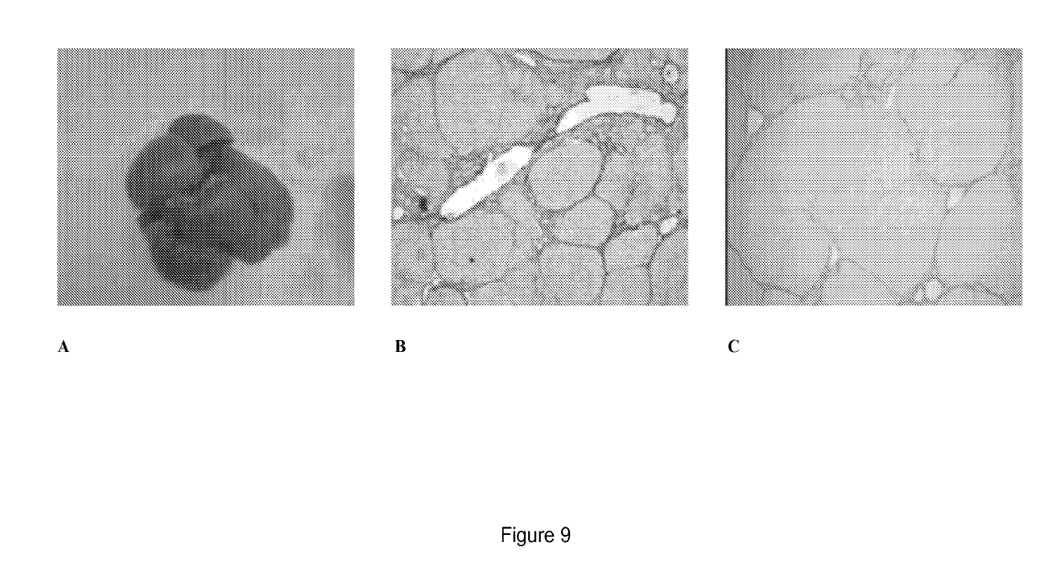

Phenotypical activation of DCs was measured. An upregulation of HLA-DR (MHC II) and CD80, was observed and found to be very similar for the three probiotic strains tested L. plantarum, L. paracasei and LGG. Phenotypical activation does not necessarily correlate with their functional activation, since the type of cytokines released can have an impact on T cell polarization. In fact the production of IL-12p70, IL-10, TNF-α and IL-12p40 by MoDCs after 24 h treatment with bacteria, was also measured. Salmonella was a strong inducer of all of the tested cytokines, while the three Lactobacilli elicited differential cytokine release (FIG. 1A). L. plantarum and LGG induced a cytokine response that was very similar to that of Salmonella, while L. paracasei induced lower levels of IL-12p70, TNF-α and IL-10 when compared to Salmonella. Thus, the only strain displaying a reduced inflammatory potential was L. paracasei CNCM 1390.

It is widely accepted that IL-10 can negatively regulate the expression of IL-12p70. To test whether an early increase in IL-10 release could impact on IL-12 production, we analyzed IL-10 release during the initial phases of DC activation (4-6 hours). The levels of IL-10 were not higher in any of the cultures of DCs with Lactobacilli during the times at which IL-12p70 was low, thereby suggesting IL-12p70 induction is delayed in comparison to Salmonella and is not controlled by IL-10 (FIG. 1B). Probiotics have a different ability to induce cytokine production by DCs.

### Example 2

### DCs T Cell Polarizing Ability

Cytokine release by DCs is important to drive the polarization of T cells towards Th1, Th2, Th17 or T regulatory cells. Given the differences observed in cytokine production we analyzed the capacity of bacteria-treated DCs to activate and polarize T cells. DCs were incubated with live bacteria and then cultured with highly purified allogeneic naïve CD4+CD45RA+ T cells. As shown in FIG. 2A, all three Lactobacilli were less potent in inducing T cell proliferation when compared to Salmonella, probably reflecting their reduced ability to upregulate surface activation markers. When we analyzed the cytokines produced by T cells we found that T cells activated with paracasei-treated DCs were affected in their ability to release IFN-γ, IL-2, IL-10 and IL-6 (FIG. 2B). In contrast, L. plantarum-treated DCs activated T cells similarly to S. typhimurium-treated cells in terms of IFN-γ release but induced less IL-10, while LGG-treated DCs induced the opposite, more IL-10 and less IFN-γ (FIG. 2B). There was no difference in IL-17 production (p>0.05). Although some probiotics (i.e. L. casei and reuteri) can induce the development of T regulatory cells, the different Lactobacilli and Salmonella displayed a similar ability to drive CD25+ Foxp3+ T regulatory cells. The difference in cytokine production reflects different T cell polarizing ability.

### Example 3

### L. paracasei Inhibits the Inflammatory Potential of DCs

Having shown that L. paracasei was the least inflammatory among the three Lactobacilli strains, we focused on this strain for further experiments. We utilized three different conditions involving the interaction between bacteria, epithelial cells and DCs. DCs were either incubated with: a. L. paracasei (LP) and S. typhimurium (SL) individually or together; b. EC supernatant for 24 h and then subsequently with each bacteria; c. supernatants of ECs pre-incubated for 24 h with L. paracasei (Sn caco LP) on the apical side and then (24 h later) with each bacterial strain. As shown in FIG. 3, L. paracasei had a strong anti-inflammatory effect on DCs both when directly co-incubated with Salmonella and indirectly when supernatants of LP-treated ECs were incubated with DCs before exposure to Salmonella. The co-incubation of DCs with LP and Salmonella significantly reduced the ability of Salmonella to induce IL-12p70 and TNF-α, while not altering its ability to promote IL-10 and IL-6 production (FIG. 3). A similar scenario was observed when DCs were first incubated with supernatants of LP-treated ECs and then infected with Salmonella. However, as we have already described [22], the incubation of DCs with unconditioned EC supernatant also reduced the ability of DCs to release IL-12p70 but not TNF in response to Salmonella. Therefore, the exposure of ECs to LP strongly inhibited the inflammatory response of Salmonella on DCs by inhibiting both IL-12p70 and TNF-α release (FIG. 3). This effect may be mediated either by LP-induced release of anti-inflammatory mediator/s by ECs, or by some component of L. paracasei that is translocated across the monolayer. The involvement of whole LP translocated across the monolayer is unlikely, as we could not detect intact bacteria from the basolateral side (not shown) and supernatants were filtered before incubation with the DCs. We could not detect any effect of LP on IL-6 or IL-10 (FIG. 3).

We then analyzed whether the factor(s) involved in the anti-inflammatory effect was a soluble mediator and could be found in the culture supernatant of L. paracasei. DCs were coincubated with Salmonella and either L. paracasei or its culture supernatant (sn LP: derived from the same amount of CFUs used for DC incubation). Interestingly, the LP supernatant alone (7% volume/volume of tissue culture medium) was extremely efficient in inhibiting the DC release of inflammatory cytokines while it was unable to alter the ability of DCs to release IL-10 or IL-12p40 (FIG. 4). When LP was extensively washed before incubation with DCs it lost the ability to inhibit the DC release of inflammatory cytokines in response to Salmonella (FIG. 4), suggesting that the anti-inflammatory effect of L. paracasei is dependent on a soluble metabolite or mediator. It is likely that this mediator(s) is not released during the limited time of LP in culture with the DCs as we could not detect LP growth during the 1 h incubation time with DCs (most likely due to the aerobic culture conditions, not shown). Further diluting the LP culture supernatant 1 to 5 (1.4% volume/volume) but also 1 to 10 (0.7% volume/volume) was still able to inhibit the release of IL-12p70 and TNF-α, indicating the high efficacy of the soluble mediator(s) (FIG. 4).

### Example 4

### Coincubation of Salmonella and L. paracasei (LP) Affects the Ability of DCs to Activate Th1 T Cells

Having shown that the coincubation of DCs with LP and Salmonella (SL) drastically reduced the ability of DCs to release IL-12p70 while preserving IL-10 production we analyzed the ability of these DCs to polarize inflammatory T cells. We treated DCs with either LP, or SL or the two together. Cells were then incubated with highly purified naïve T cells and cytokine release in culture supernatants was tested. As shown in FIG. 5 (see the situation a), T cells activated with DCs that were incubated with both Salmonella and L. paracasei were highly impaired in their ability to release IL-2, IL-10, IL-6 and IFN-γ. We could not observe any difference in IL-17, IL-13 or IL-5 suggesting that LP+SL treated DCs were still capable of inducing Th17 or Th2 polarization (FIG. 5).

### Example 5

### DCs Incubated with Supernatants of L. paracasei Treated ECs are Affected in their Ability to Drive Th1 T Cells

DCs incubated with supernatants of LP-treated ECs are affected in their ability to release IL12-p70 and TNF-α in response to S. typhimurium. Consequently, we evaluated whether this had an impact on Th1 T cell polarization. To accomplish this, DCs were pre-incubated with supernatants from either untreated (sn caco: situation b) or paracasei-treated ECs (sn caco LP: situation c) for 24 h, and then with either LP or Salmonella for an additional 24 h before incubation with naïve T cells for 5 days. As shown in FIG. 4, the preincubation of DCs with LP-treated-EC supernatants prior to Salmonella infection, drastically reduced the DC's ability to activate T cells and drive their polarization to Th1 T cells as evidenced by a decrease in IFN-γ, IL-2 and IL-6 production. There was no difference in IL-17 and IL-13 production (p>0.05) while IL-10 and IL-5 levels were also reduced in culture supernatants (FIG. 5). This indicates that the incubation of ECs with L. paracasei has a strong effect on the ability of DCs to activate T cells in response to Salmonella; in particular, this impairment regards their ability to drive Th1 T cells.

### Example 6

### DCs Incubated with Supernatants of L. paracasei Treated ECs are Affected in their Ability to Drive Th1 T Cells

DCs incubated with supernatants of LP-treated ECs are affected in their ability to release IL12-p70 and TNF-α in response to Salmonella. Consequently, we evaluated whether this had an impact on Th1 T cell polarization. To accomplish this, DCs were pre-incubated with supernatants from either untreated (sn caco: situation b) or paracasei-treated ECs (sn caco LP: situation c) for 24 h, and then with either LP or Salmonella for an additional 24 h before incubation with naïve T cells for 5 days. As shown in FIG. 5, the preincubation of DCs with LP-treated-EC supernatants prior to Salmonella infection, drastically reduced the DC's ability to activate T cells and drive their polarization to Th1 T cells as evidenced by a decrease in IFN-γ, IL-2 and IL-6 production. There was no difference in IL-17 and IL-13 production (p>0.05) while IL-10 and IL-5 levels were also reduced in culture supernatants (FIG. 5). This indicates that the incubation of ECs with L. paracasei has a strong effect on the ability of DCs to activate T cells in response to Salmonella.

### Example 7

### Results of the In Vitro Test Correlate with the Results In Vivo in a Murine Model

We next compared the activity of the three Lactobacilli in protecting mice against an acute model of colitis. We chose the DSS colitis model as it provokes a strong inflammatory response that is primarily mediated by DCs. Mice were pre-treated i.g. for 7 days with 10¹⁰ CFUs of either L. plantarum, LGG or L. paracasei, or with PBS as a control. Then mice received for 5 days 2% DSS in the drinking water and the development of colitis was followed over time by measurement of body weight, stool consistency and presence of blood in the feces. We found that L. plantarum and LGG, consistent with their ability to strongly activate DCs, were not only ineffective in protecting against colitis, but were in fact detrimental. Indeed, LGG- and L. plantarum-treated mice displayed an increased disease activity index (DAI) and all died between 10 and 12 days from DSS administration (FIG. 6). In contrast, mice receiving L. paracasei although displaying a similar weight loss as PBS-DSS treated controls, showed a delay in colitis development and a reduced severity of disease (as shown by reduced DAI in FIG. 6B). Therefore the in vitro activity of probiotics is predictive of their efficacy in vivo.

### Example 8

### L. paracasei Activity in a Mouse Model of CCl₄-Induced Liver Fibrosis

### Effect on Intestinal Permeability

The double sugar test allows to analyze the intestinal permeability by monitoring the amount of the sugar probes in the urine after 5 hours.

Data from this test have shown that the induction of fibrosis by CCl₄ increases the intestinal permeability that is restored by the treatment with L. paracasei (see the recovery rates of both mannitol (P<0.05) and lactulose).

### Effects on the Intestinal Microflora

The assessment of intestinal microflora in rat's colon, carried by DGGE, showed a healthy bacterial flora characterized mainly by: Clostridium (species not further characterized) and one Lactobacillus species belonging to the group of Lactobacillus acidophilus and Lactobacillus intestinalis. In rats where fibrosis was induced by CCl₄ an increase in bacteria of the genus Clostridium (presumably papirosolvens species) was observed, while in almost all rats treated with L. paracasei 21060 a decrease in bacteria belonging to this genus was observed.

### Serum Levels of AST and ALT

AST (alanine amminotransferase), ALT (aspartate amminotransferase), γ-GT (γ-glutamminil-transferase, ALP (alkaline phosphatase) and total bilirubin are biochemical markers of hepatic injury.

Serum levels of AST, ALT, ALP and total bilirubin are increased in rats with liver fibrosis compared with control rats (p<0.05); these amounts were normalized by treatment with L. paracasei (Tab.2).

TABLE 2
Evaluation of biochemical parameters in serum.

| | | rats treated | rats treated |
|---|---|---|---|
| | Normal control | with CCl₄ | with CCl₄ + probiotic |
| ALT | 51.20 ± 9.3 | 114.4 ± 35.7* | 40.6 ± 17.6 |
| AST | 72.50 ± 13.9 | 183.4 ± 61.5* | 117.8 ± 21.7 |
| ALP | 56 ± 25.4 | 467.6 ± 273* | 365.6 ± 68.4* |
| γ-GT | 4.2 ± 1.54 | 6.00 ± 1.60 | 4.4 ± 1.50 |
| Total bilirubin | 0.24 ± 0.05 | 0.42 ± 0.08* | 0.32 ± 0.08* |

(*p < 0.05 vs normal)

### Serum Levels of TNF-α and IL-10

IL-10 levels were lower in the group of rats with fibrosis than in the control group and significantly increased after treatment with probiotic (p<0.05; Tab.3). TNF-α levels increased during hepatic fibrogenesis caused by CCl₄ in rats compared with measured values of the control group and were significantly reduced following treatment with L. paracasei (p<0.05; Tab.3 and FIG. 7).

TABLE 3
Serum levels of mouse/rat TNF-α and mouse/rat
IL-10 specific by quantitative ELISA test.

| | Control (normal) | CCl₄ rats | CCl₄ + probiotic |
|---|---|---|---|
| IL-10 | 90 ± 6.9 | 126.5 ± 21.4 | 164.1 ± 26.8 * ** |
| TNF-α | 47.4 ± 12.3 | 44.1 ± 12.9 | 9.1 ± 13.8 * |

(* P < 0.05 vs Normal,
** p < 0.05 vs CCl₄)

mRNA expression of anti-inflammatory and pro-inflammatory cytokines in rat liver tissue.

In rats subjected to induction with CCl₄ was found, compared with rats in the control group, increased levels of mRNA for TNF-α, TGF-β1, TLR2 and TLR4, and a decrease in the mRNA levels for the IL-10. The group of animals who received the probiotic has showed instead a decrease in the levels of mRNA for TNF-α, TGF-β1, TLR2, TLR4 and iNOS and an increase of mRNA levels of IL-10 mRNA and eNOS, compared with control rats. The gene expression levels of IL-1β were not modified following treatment with L. paracasei (FIGS. 8 A and B). TGF-β1 and TNF-α are useful markers of hepatotoxicity that leads to hepatic damages (Li-Juan Zhang et al. World Journal Gastroenterol 2004; 10(1): 77-81). Lowering of IL-10 levels in fibrotic rats is consistent with the role of this cytokine which acts as a modulator of the inflammatory response by reducing the hepatotoxic effect.

### Histological Analysis of Liver Tissue

The histological analysis revealed a normal architecture of liver tissue in rats belonging to the control group. In rats treated with CCl₄ for 7 weeks is observable a marked fibrosis with altered tissue architecture, formation of large fibrous septa, pseudolobi separation (see FIG. 9A) and collagen accumulation (FIG. 9B). These qualitative and quantitative histopathological changes are significantly reduced in liver sections of rats that received daily probiotic (FIG. 9C). The reduction of fibrosis is also confirmed by quantitative data showing a reduced rate of collagen deposition in the liver of rats treated with L. paracasei (4.2±0.5 *; * (P<0.05) vs CCl₄) compared to the control group (9.2±2.9). The calculation of these values was obtained by electronic image analysis by a workstation consisting of a computer linked to a microscope with camera. Tissue samples were amplifyed 10× with a video camera Sony 3CCD. The system automatically indicates the number of collagen fibers present in the sample.

1. A method for selecting a probiotic strain having bowel anti-inflammatory properties comprising the steps of:
co-incubating isolated Dendritic Cells (DCs) in the presence of an aliquot of a cell-depleted conditioned culture medium where a putative probiotic strain has been grown, together with a strong bacterial antigen,
detecting the production levels of the following cytokines: IL-12p70, TNF-α and IL-10 produced by DCs upon co-incubation,
selecting a probiotic strain where a decrease of IL-12p70 and TNF-α levels and substantially no alteration of IL-10 levels, with respect to the cytokine level measured in the absence of the putative probiotic, is detected.

2. (canceled)

3. The method according to claim 1, wherein said strong bacterial antigen is selected in the group consisting of: S. typhimurium, E. coli, commensal bacteria and antigens thereof.

4. The method according to claim 1, wherein cytokine measurement is carried out by an immunoassay.

5. The method according to claim 4 wherein said immunoassay is an ELISA assay.

6. The method according to claim 1, wherein said DCs are isolated from peripheral blood mononuclear cells (PBMC).

7. The method according to claim 1, wherein said isolated DCs are human.

8. The method according to claim 1 wherein, said putative probiotic strains are selected from the group consisting of: Lactobacillus johnsonii, Lactobacillus reuterii, Lactobacillus paracasei, Lactobacillus casei, Lactobacillus animalis, Lactobacillus ruminis, Lactobacillus acidophilus, Lactobacillus rhamnosus, Lactobacillus fermentum, Lactobacillus delbrueckii subsp. Lactis, Bifidobacterium spp., Bifidobacterium bifidum, Bifidobacterium longum, Bifidobacterium pseudolongum, Bifidobacterium infantis, Bifidobacterium adolescentis, and Bifidobacterium lactis.

9. A method for preparing a probiotic composition comprising the steps of:
a. selecting a probiotic strain according to the method of claim 1;
b. growing said probiotic strain in a suitable culture medium up to an exponential growth phase;
c. depleting bacterial cells from the probiotic strain culture medium;
d. admixing said cell depleted culture medium with excipients or diluents suitable for an oral administration composition.

10. (canceled)

11. The method of claim 10 wherein said depletion is carried out by centrifugation, sedimentation, and/or filtration.

12. The method of claim 9, wherein said probiotic strain is a Lactobacillus paracasei CNCM I-1390.

13-24. (canceled)

25. A method for the treatment of a chronic liver disease in a mammal comprising administering to a mammal in need thereof an effective amount of a Lactobacilli paracasei strain CNCM I-1390 or supernatant thereof in a oral composition.

26. The method according to claim 25 wherein said chronic liver disease is liver fibrosis due to a metabolic or toxic cause.


SEQUENCE LISTING (16 sequences)

SEQ ID NO: 1 - DNA, 20 bp, Rattus norvegicus
  primer_bind (1)..(20)
aaatgggctccctctcatca

SEQ ID NO: 2 - DNA, 20 bp, Rattus norvegicus
  primer_bind (1)..(20)
tccttagggcaagggctctt

SEQ ID NO: 3 - DNA, 21 bp, Rattus norvegicus
  primer_bind (1)..(21)
tggctcagcactgctatgttg

SEQ ID NO: 4 - DNA, 22 bp, Rattus norvegicus
  primer_bind (1)..(22)
tccagagggtcttcagcttctc

SEQ ID NO: 5 - DNA, 21 bp, Rattus norvegicus
  primer_bind (1)..(21)
acttgggctgtccagatgaga

SEQ ID NO: 6 - DNA, 20 bp, Rattus norvegicus
  primer_bind (1)..(20)
gcctgcagtgcagctgtcta

SEQ ID NO: 7 - DNA, 20 bp, Rattus norvegicus
  primer_bind (1)..(20)
cgcatagagacatccaaagg

SEQ ID NO: 8 - DNA, 20 bp, Rattus norvegicus
  primer_bind (1)..(20)
ttctcacccagtcctcattc

SEQ ID NO: 9 - DNA, 20 bp, Rattus norvegicus
  primer_bind (1)..(20)
cccttgacatcagcaagaac

SEQ ID NO: 10 - DNA, 20 bp, Rattus norvegicus
  primer_bind (1)..(20)
acaggagttcacaggagcag

SEQ ID NO: 11 - DNA, 20 bp, Rattus norvegicus
  primer_bind (1)..(20)
ctcaggttctgtgtgtttgg

SEQ ID NO: 12 - DNA, 20 bp, Rattus norvegicus
  primer_bind (1)..(20)
ggatttgctgctctgtaggt

SEQ ID NO: 13 - DNA, 20 bp, Rattus norvegicus
  primer_bind (1)..(20)
agcgagttgtggattgttct

SEQ ID NO: 14 - DNA, 19 bp, Rattus norvegicus
  primer_bind (1)..(19)
cttcgggcttcaggttatt

SEQ ID NO: 15 - DNA, 20 bp, Rattus norvegicus
  primer_bind (1)..(20)
gtcataccaggaaatgagct

SEQ ID NO: 16 - DNA, 20 bp, Rattus norvegicus
  primer_bind (1)..(20)
gccaaaagggtcatcatctc